(19) United States
(12) Patent Application Publication
(10) Pub. No.: US 2026/0104171 A1
(43) Pub. Date: Apr. 16, 2026

(54) CLEAN ROOM FACILITY

(71) Applicant: HITACHI GLOBAL LIFE SOLUTIONS, INC., Tokyo (JP)

(72) Inventors: Nobuhiro IMAGUCHI, Tokyo (JP); Noritoshi NISHIMURA, Tokyo (JP); Kazuhito MATSUZAKI, Tokyo (JP)

(21) Appl. No.: 19/111,366

(22) PCT Filed: Feb. 10, 2023
(86) PCT No.: PCT/JP2023/004512

§ 371 Date: Mar. 13, 2025

(51) Int. Cl. F24F 3/167; A61L 2/208; A61L 103/75

(52) U.S. Cl. CPC F24F 3/167; A61L 2/208; A61L 2103/75; A61L 2202/11; A61L 2202/15; F24F 2221/22

(57) ABSTRACT

To provide a clean room facility that appropriately performs air conditioning of a clean room. The clean room facility includes: a front room and a work room that are clean rooms provided inside a large room; an air handling unit configured to adjust a temperature of air supplied to the large room; a chamber provided as one common space above a ceiling of the front room and the work room and configured to guide air from the large room; an air supply fan; and a duct shaft. The duct shafts include the duct shaft that does not communicate with the chamber, and air is guided from the front room and the work room to the duct shaft not communicating with the chamber, and is exhausted through the duct shaft.

### TECHNICAL FIELD

The present invention relates to a clean room facility.

### BACKGROUND ART

A clean room having high air cleanliness is used in regenerative medicine, pharmaceutical manufacturing, and the like. Regarding such a clean room, for example, PTL 1 discloses that “the fan controlled based on the detection value of the pressure sensor and the fan controlled at a constant speed out of the first fan and the second fan are switchable”.

### CITATION LIST

### Patent Literature

PTL 1: WO2022/254705

### SUMMARY OF INVENTION

### Technical Problem

In the technique described in PTL 1, a predetermined clean room can be switched from one of a positive pressure room and a negative pressure room to the other, but there is room for improvement in terms of air conditioning of the clean room.

Therefore, an object of the invention is to provide a clean room facility that appropriately performs air conditioning of a clean room.

### Solution to Problem

In order to solve the above problems, the clean room facility according to the invention includes: a plurality of clean rooms provided inside a predetermined room; a first air handling unit configured to adjust a temperature of air supplied to the predetermined room; a chamber provided as one common space above a ceiling of the plurality of clean rooms and configured to guide air from the predetermined room; an air supply fan provided in each of the plurality of clean rooms and configured to supply air from the chamber to the clean room; and a duct shaft provided outside a side wall of at least one of the clean rooms. Among the duct shafts, the duct shafts facing the predetermined room include a plurality of duct shafts whose upper ends are closed so as not communicate with the chamber, and further, provided with maintenance covers, air is guided from the clean room to the duct shaft not communicating with the chamber, and is exhausted through the duct shaft, and an exhaust destination when the air is exhausted from the clean room through the duct shafts is the predetermined room.

### Advantageous Effects of Invention

According to the invention, it is possible to provide the clean room facility that appropriately performs air conditioning of the clean room.

### BRIEF DESCRIPTION OF DRAWINGS

FIG. 1 is a diagram illustrating a layout of rooms of a clean room facility according to a first embodiment.

FIG. 2 is a schematic cross-sectional view illustrating the clean room facility according to the first embodiment.

FIG. 3 is a diagram illustrating another example related to processing during sterilization of a work room and a front room in the clean room facility according to the first embodiment.

FIG. 4 is a diagram illustrating another example related to processing during sterilization of the work room and the front room in the clean room facility according to the first embodiment.

FIG. 5 is a schematic cross-sectional view illustrating a clean room facility according to a first modification of the first embodiment.

FIG. 6 is a schematic cross-sectional view illustrating a clean room facility according to a second modification of the first embodiment.

FIG. 7 is a schematic cross-sectional view illustrating a clean room facility according to a second embodiment.

FIG. 8 is a schematic cross-sectional view showing a clean room facility according to a comparative example.

### DESCRIPTION OF EMBODIMENTS

### First Embodiment

### Configuration of Clean Room Facility

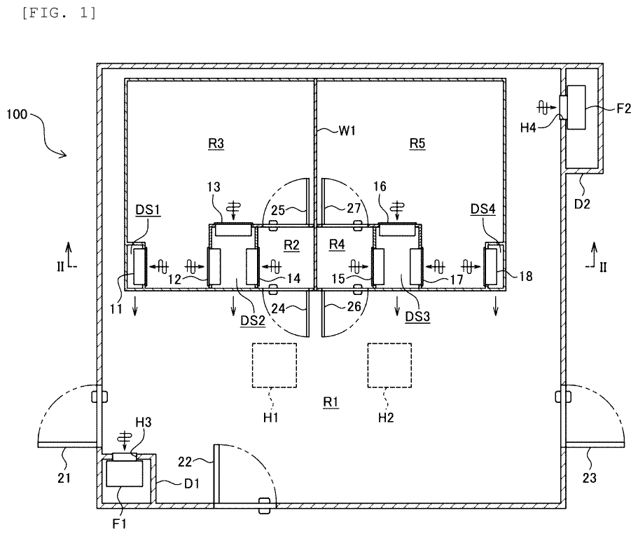

FIG. 1 is a diagram illustrating a layout of rooms of a clean room facility 100 according to a first embodiment.

In FIG. 1, directions in which air flows are indicated by arrows. The clean room facility 100 is a facility that adjusts a temperature, a room pressure, cleanliness, and the like of a plurality of clean rooms such as front rooms R2 and R4 and work rooms R3 and R5. Such the clean room facility 100 is used, for example, for cell culture processing and production of sterile preparations (vaccines, injections, eye drops, etc.). In an example in FIG. 1, the clean room facility 100 includes the front rooms R2 and R4 (clean rooms) and the work rooms R3 and R5 (clean rooms) in addition to a large room R1 (predetermined room).

The large room R1 is a relatively wide room in which predetermined preprocessing, analysis, and the like are performed. Although not illustrated, an operation panel, a monitoring device, or a utility device for equipment used in the work rooms R3 and R5 may be provided in the large room R1. Hereinafter, a case where the large room R1 is a clean room will be described, but the large room R1 may be a normal room. The normal room is, for example, a room where cleanliness is not managed, with respect to the clean room.

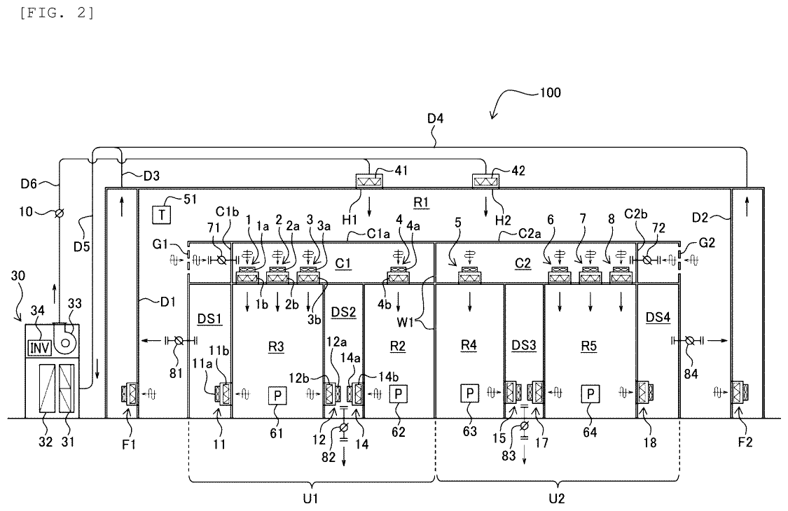

As illustrated in FIG. 1, doors 21 to 23 that are opened and closed when a person enters and exits or when a device is carried in and out are provided at predetermined locations (three locations in the example in FIG. 1) of the large room R1. In addition, on a ceiling of the large room R1, air outlets H1 and H2 (first air outlets) are provided from which air whose temperature is adjusted by an air handling unit 30 (first air handling unit: see FIG. 2) is blown out.

Ducts D1 and D2 are provided at predetermined locations (two corners in FIG. 1) of the large room R1. The duct D1 is provided with an air inlet H3 (second air inlet) through which air is suctioned from the large room R1 toward the air handling unit 30 (first air handling unit: see FIG. 2). Similarly, the other duct D2 is provided with an air inlet H4. As illustrated in FIG. 1, a fan F1 is installed in the air inlet H3 of the duct D1. The fan F1 is a blower that sends air from the large room R1 to the air handling unit 30 (see FIG. 2) via the duct D1. Similarly, a fan F2 is installed in the other duct D2. The fans F1 and F2 are not essential, and when the fans F1 and F2 are not provided, for example, a duct D3 may be connected to a wall near the fan F1 to replace the duct D1, or the fan F1 may be simply not provided.

Inside the large room R1 (predetermined room), the front rooms R2 and R4 and the work rooms R3 and R5 are provided as a plurality of clean rooms. The front room R2 is used, for example, as an airlock for preventing sample contamination. The front room R2 may be used for undressing, clothing, or predetermined preprocessing. In the example in FIG. 1, a person can enter and exit between the front room R2 and the large room R1 via a door 24. Cleanliness of the front room R2 may be higher than cleanliness of the large room R1, or may be equal to the cleanliness of the large room R1. That is, the cleanliness of the front room R2 is equal to or higher than the cleanliness of the large room R1.

The work room R3 is a clean room in which a sample is prepared. Examples of the “sample” include, but are not limited to, cells and sterile preparations. Since the sample is adjusted in the work room R3, cleanliness of the work room R3 is higher than the cleanliness of the front room R2 and the large room R1. When the large room R1 is a clean room, the cleanliness of the work room R3 is equal to or higher than the cleanliness of the front room R2 and the large room R1. A person can enter and exit between the front room R2 and the work room R3 via a door 25.

Regarding a room pressure of each clean room, for example, a room pressure of the front room R2 may be lower than a room pressure of the large room R1 or the work room R3 in order to prevent sample contamination. Accordingly, it is possible to prevent dust from entering the work room R3 from the large room R1 via the front room R2 in a process of opening and closing the doors 24 and 25 and people coming and going. It is also possible to prevent the sample (aerosol) from flowing out from the work room R3 to the large room R1 via the front room R2. The room pressures of the large room R1 and the work room R3 may be equal to each other, or one may be higher than the other. In addition, when the room pressure of the front room R2 is higher than the room pressure of the large room R1 or the work room R3, the front room R2 functions as an airlock. The same applies to the room pressure of the other front room R4 and work room R5.

In the example in FIG. 1, the front room R2 is adjacent to another front room R4 via a wall W1. Similarly, the work room R3 is adjacent to another work room R5 via the wall W1. That is, the front rooms R2 and R4 are partitioned by the wall W1, and the work rooms R3 and R5 are also partitioned by the wall W1.

The clean room facility 100 includes duct shafts DS1, DS2, DS3, and DS4 illustrated in FIG. 1 in addition to the above-described components. The duct shaft DS1 is a flow path that guides air from the work room R3 to the large room R1, and extends in a tubular shape in a vertical direction (see also FIG. 2). The duct shaft DS1 is provided in a gap between the work room R3 (predetermined clean room) and the large room R1 (predetermined room). In the example in FIG. 1, the duct shaft DS1 is provided at one corner of the work room R3. From another viewpoint, the duct shaft DS1 is provided outside a side wall of the work room R3.

A part of the side wall of the work room R3 may form a part of the duct shaft DS1. The duct shaft DS1 may be formed by fitting a cylindrical duct (not illustrated) into the gap between the work room R3 and the large room R1. The same applies to the other duct shafts DS2, DS3, and DS4. As illustrated in FIG. 1, the duct shaft DS1 is provided with a fan filter unit 11 for exhausting air from the work room R3 to the large room R1.

Another duct shaft DS2 is a flow path that guides air from the front room R2 or the work room R3 to the large room R1, and extends in a tubular shape in the vertical direction (see also FIG. 2). As illustrated in FIG. 1, the duct shaft DS2 is provided in a gap between the front room R2 and the work room R3 (that is, between the clean rooms). From another viewpoint, it can be said that the duct shaft DS2 is provided in a gap between the work room R3 (predetermined clean room) and the large room R1 (predetermined room). The duct shaft DS2 is provided outside a side wall of the front room R2 and outside the side wall of the work room R3. The duct shaft DS2 is provided with fan filter units 12, 13 for exhausting air from the work room R3 to the large room R1 and a fan filter unit 14 for exhausting air from the front room R2 to the large room R1.

The front room R4, the work room R5, and the duct shafts DS3 and DS4 illustrated in FIG. 1 are arranged substantially symmetrically in a plan view with respect to the front room R2, the work room R3, and the duct shafts DS1 and DS2 with respect to the wall W1, and thus description thereof is omitted. A layout and the number of clean rooms in FIG. 1 are merely examples, and the layout may be rotated by 90°, for example, but the invention is not limited thereto.

FIG. 2 is a schematic cross-sectional view illustrating the clean room facility 100.

Although FIG. 2 is roughly a cross-sectional view taken along a line II-II in FIG. 1, the air handling unit 30 is also illustrated in FIG. 2 in addition to the ducts D1 and D2 (see also FIG. 1) of the large room R1 in order to clearly illustrate a flow of air.

As described above, the ducts D1 and D2 are air conduits through which the air exhausted from the large room R1 flows, and extend in the vertical direction. Then, air flowing from the large room R1 sequentially through the ducts D1 and D3 and air flowing from the large room R1 sequentially through ducts D2 and D4 are merged, and merged air is guided to a suction side of the air handling unit 30 via a duct D5.

As illustrated in FIG. 2, the clean room facility 100 includes the air handling unit 30 (first air handling unit), chambers C1 and C2, a temperature sensor 51, and pressure sensors 61 to 64. In addition to the above-described configuration, the clean room facility 100 includes dampers 71 and 72 (first damper), other dampers 81 to 84 (second damper), fan filter units 1 to 8 on an air supply side, and fan filter units 11 to 18 on an exhaust side.

The air handling unit 30 is a device that adjusts a temperature, humidity, and the like of air supplied to the large room R1 (predetermined room). As illustrated in FIG. 2, the air handling unit 30 includes a filter 31, a cooling coil 32, a fan 33, and an inverter 34. The filter 31 collects dust from the air flowing from the large room R1 through the duct D5 or the like toward the cooling coil 32. The cooling coil 32 is a heat exchanger that exchanges heat between air passed through the filter 31 and a refrigerant flowing through a heat transfer pipe (not illustrated). The heat exchanger may be provided with a humidifying function to adjust the humidity of the air supplied to the large room R1. The fan 33 is a blower that sends the air heat-exchanged by the cooling coil 32 into the large room R1 through the duct D6. The inverter 34 drives a motor (not illustrated) of the fan 33 in a predetermined manner.

As illustrated in FIG. 2, a blowing side of the fan 33 and the air outlets H1 and H2 on a ceiling of the large room R1 are connected via the duct D6. The air outlet H1 is provided with a filter 41 for collecting dust from air (the same applies to the other air outlet H2). When the large room R1 is used as a normal room instead of a clean room, it is not particularly necessary to install the filters 41 and 42.

The air cooled by the air handling unit 30 flows through the duct D6, and is blown out to the large room R1 through the air outlets H1 and H2. As illustrated in FIG. 1, a damper 10 is installed in the duct D6. For example, the damper 10 is set to a predetermined opening degree during a test operation of the air handling unit 30, and is maintained at the predetermined opening degree during a subsequent air conditioning operation.

The temperature sensor 51 is a sensor that detects a temperature of the large room R1, and is installed at a predetermined location in the large room R1. A detection value of the temperature sensor 51 is used to control the air handling unit 30. In addition, although not illustrated, a humidity sensor may be provided in addition to the temperature sensor 51, and a temperature and humidity of the large room R1 may be adjusted by the air handling unit 30.

The pressure sensor 61 is a sensor that detects a room pressure of the work room R3, and is installed in the work room R3. A detection value of the pressure sensor 61 is used to control the fan filter units 11 to 13 and the like on an exhaust side of the work room R3. The fan filter unit 13 (see FIG. 1) on a rear side of the duct shaft DS2 and a fan filter unit 16 (see FIG. 1) on a rear side of another duct shaft DS3 are not illustrated in FIG. 2.

The pressure sensor 62 is a sensor that detects a room pressure of the front room R2, and is installed in the front room R2. A detection value of the pressure sensor 62 is used to control the fan filter unit 14 and the like on an exhaust side of the front room R2. The same applies to the remaining pressure sensor 63 of the front room R4 and the remaining pressure sensor 64 of the work room R5.

The chamber C1 illustrated in FIG. 2 is a space above a ceiling of the work room R3 and the front room R2. That is, the chamber C1 is provided as one common space above the ceiling of a plurality of clean rooms such as the work room R3 and the front room R2. Another chamber C2 illustrated in FIG. 2 is a space above a ceiling of the front room R4 and the work room R5. These two chambers C1 and C2 are adjacent to each other via the wall W1. The wall W1 partitions the chambers C1 and C2, partitions the front rooms R2 and R4, and partitions the work rooms R3 and R5 as described above (see FIG. 1).

A grill G1 illustrated in FIG. 2 is a member that guides air in the large room R1 to the damper 71 while preventing the damper 71 from being exposed to the large room R1. The grill G1 may have a configuration in which a plurality of blades (louvers) are arranged in parallel at predetermined intervals, or may have a mesh-shaped configuration. Gaps between the blades of the grill G1 (a plurality of holes in the case of a mesh) function as a “first air inlet” through which air is suctioned from the large room R1 (predetermined room) toward the chamber C1. The “first air inlet” is provided near the chamber C1. The same applies to another grill G2 on a right side of a paper in FIG. 2.

In FIG. 2, the grill G1 is illustrated on the left side of the paper, and the other grill G2 is illustrated on a right side of the paper, but the grills G1 and G2 may be provided on a front side on which doors 24 and 26 (see FIG. 1) are provided.

The damper 71 (first damper) switches between communication and interruption between the large room R1 (predetermined room) and the chamber C1. The damper 71 is provided at a position facing the grill G1 on an air inlet side of the chamber C1. Similarly, the damper 72 is provided on an air inlet side of the other chamber C2. As these dampers 71 and 72, non-leak dampers (airtight dampers) having high airtightness in a closed state may be used. Accordingly, when the work rooms R3 and R5 and the front rooms R2 and R4 are sterilized, it is possible to prevent the sterilization gas from flowing into the large room R1 by closing the dampers 71 and 72.

The dampers 71 and 72 are maintained in an open state during normal use of the clean room facility 100. That is, during normal use of the clean room facility 100, each of the chambers C1 and C2 communicates with the large room R1. The air in the large room R1 is guided to the chamber C1 sequentially through the grill G1 and the damper 71 (open state). Similarly, the air in the large room R1 is guided to the other chamber C2 sequentially through the grill G2 and the damper 72 (open state).

The chamber C1 includes the ceiling of the work room R3 and the front room R2, an upper plate C1a, and a side plate C1b. The upper plate C1a is higher than the ceiling of the work room R3 and the front room R2, and is substantially parallel to the ceiling. The side plate C1b is a plate connecting an edge of the ceiling of the work room R3 or the front room R2 and an edge of the upper plate C1a, and extends in the vertical direction. The same applies to the other chamber C2.

Hereinafter, a structure including a plurality of clean rooms having a common chamber is referred to as a “clean unit”. In the example in FIG. 2, a structure including the work room R3 and the front room R2 having the common chamber C1 above the ceiling is referred to as a first clean unit U1. Further, a structure including the front room R4 and the work room R5 having the common chamber C2 above the ceiling is referred to as a second clean unit U2. The first clean unit U1 and the second clean unit U2 are adjacent to each other via the wall W1, but may be separated from each other.

The fan filter units 1 to 3 illustrated in FIG. 2 are devices that supply air from the chamber C1 to the work room R3, and are embedded in the ceiling of the work room R3. The fan filter unit 1 includes an air supply fan la and a filter 1b. The air supply fan la is a blower that supplies air from the chamber C1 to the work room R3 (clean room).

The filter 1b collects dust from the air flowing from the air supply fan la to the work room R3, and is provided on a blowing side of the air supply fan 1a. Examples of the filter 1b include high efficiency particulate air filter (HEPA) and ultra low penetration air filter (ULPA). The remaining fan filter units 2 and 3 used to supply air to the work room R3 have a similar configuration.

The fan filter unit 4 is a device that supplies air from the chamber C1 to the front room R2. The fan filter unit 4 includes an air supply fan 4a and a filter 4b, and is embedded in the ceiling of the front room R2. Thus, the “air supply fan” is provided in each of the plurality of “clean rooms”. The other fan filter units 5 to 8 on the air supply side have a similar configuration.

The fan filter unit 11 illustrated in FIG. 2 is a device that exhausts air from the work room R3, and includes an exhaust fan 11a and a filter 11b. The exhaust fan 11a is a blower that exhausts air from the work room R3, and is installed on the duct shaft DS1. That is, an opening is provided at a predetermined location facing the work room R3 in the duct shaft DS1, and the fan filter unit 11 is fitted into the opening. The filter 11b collects dust from air flowing from the work room R3 toward the exhaust fan 11a, and is provided on a suction side of the exhaust fan 11a. Other fan filter units 12 to 18 on the exhaust side have a similar configuration.

As illustrated in FIG. 2, an upper end of the duct shaft DS1 is closed and does not communicate with the chamber C1. The duct shaft DS1 communicates with the work room R3 via the exhaust fan 11a. Air is guided from the work room R3 (clean room) to the duct shaft DS1 that does not communicate with the chamber C1, and is exhausted through the duct shaft DS1. In other words, the air is exhausted from the work room R3 (clean room) communicating with the duct shaft DS1 through the duct shaft DS1 not communicating with the chamber C1. In the example in FIG. 2, an exhaust destination when air is exhausted from the work room R3 through the duct shaft DS1 is the large room R1 (predetermined room). The same applies to the exhaust through the other duct shafts DS2, DS3, and DS4.

The damper 81 (second damper) illustrated in FIG. 2 switches between communication and interruption between the duct shaft DS1 and the large room R1 (predetermined room), and is provided on a side of the duct shaft DS1 facing the large room R1. The damper 82 (second damper) illustrated in FIG. 2 switches between communication and interruption between the duct shaft DS2 and the large room R1. The same applies to the other dampers 82, 83, and 84. As these dampers 81 to 84, non-leak dampers (airtight dampers) having high airtightness in a closed state may be used. Accordingly, for example, when the work rooms R3 and R5 and the front rooms R2 and R4 are sterilized, it is possible to prevent the sterilization gas from flowing into the large room R1 by closing the dampers 81 to 84. The dampers 81 to 84 are maintained in an open state during normal use of the clean room facility 100.

The fan filter unit 12 illustrated in FIG. 2 and the fan filter unit 13 illustrated in FIG. 1 are devices for exhausting air from the work room R3, and are installed on the duct shaft DS2. Another fan filter unit 14 is a device that exhausts air from the front room R2, and is installed on the duct shaft DS2.

As described above, the duct shaft DS2 is provided in the gap between the work room R3 and the front room R2. Since the upper end of the duct shaft DS2 is also closed, the duct shaft DS2 does not communicate with the chamber C1. The duct shaft DS2 communicates with the work room R3 via an exhaust fan 12a and the like, and also communicates with the front room R2 via another exhaust fan 14a. When these exhaust fans 12a to 14a are driven, the air in the work room R3 and the front room R2 is exhausted sequentially through the duct shaft DS2 and the damper 82 (open state) to the large room R1 (see also FIG. 1). A downward arrow near the damper 82 in FIG. 2 indicates a flow of air toward the large room R1.

The fan filter units 1 to 8 on the air supply side and the fan filter units 11 to 18 on the exhaust side are controlled by a control device (not illustrated). The control device may be built in each of the fan filter units 1 to 8, and 11 to 18, or a plurality of fan filter units may be connected to one control device via wiring.

A rotation speed of the exhaust fans 11a, 12a, and 13a (see also FIG. 1) is controlled, for example, to maintain a room pressure of the work room R3 at a predetermined set pressure (target pressure). A rotation speed of the air supply fans 1a, 2a, and 3a may be constant, or may be appropriately adjusted based on the room pressure of the work room R3. The same applies to room pressure control of the remaining front rooms R2 and R4 and work room R5. In addition, since the clean room facility 100 has a substantially symmetrical configuration with respect to the wall W1, description of a configuration related to air conditioning of the front room R4 and the work room R5 will be omitted.

### Comparative Example

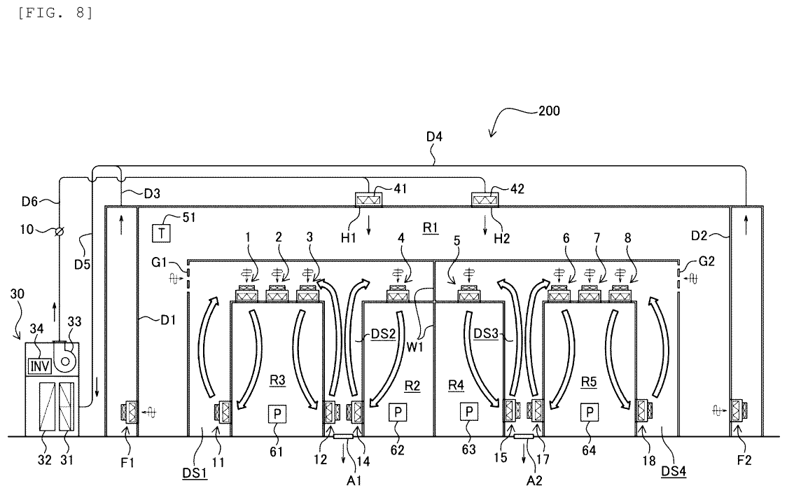

FIG. 8 is a schematic cross-sectional view illustrating a clean room facility 200 according to a comparative example.

The comparative example in FIG. 8 is different from the first embodiment (see FIG. 2) in that no damper is provided, the duct shafts DS1 and DS2 communicate with the chamber C1, and the duct shafts DS3 and DS4 communicate with the other chamber C2. The comparative example in FIG. 8 is different from the first embodiment in that the duct shafts DS1 and DS4 do not communicate with the large room R1. The comparative example in FIG. 8 is different from the first embodiment (see FIG. 2) in that the duct shaft DS2 communicates with the large room R1 via an opening A1, and the remaining duct shaft DS3 communicates with the large room R1 via another opening A2.

In the configuration of the comparative example in FIG. 8, for example, air flowing into the duct shaft DS1 from the work room R3 is returned to the chamber C1 as the fan filter unit 11 on the exhaust side is driven. As the fan filter units 12 to 14 on the exhaust side is driven, a part of the air flowing out from the work room R3 and the front room R2 to the duct shaft DS2 is exhausted to the large room R1 through the opening A1, and the remaining air is returned to the chamber C1 through the duct shaft DS2.

Then, as indicated by white arrows in FIG. 8, for example, the air absorbed in the work room R3 is returned to the chamber C1 through the duct shaft DS1, and most of the air is supplied to the work room R3 by the fan filter units 1 to 3 on the air supply side. If such air circulation (closed loop) occurs, a flow of conditioned air (air cooled by the air handling unit 30) guided from the large room R1 to the chamber C1 when the air is supplied to the work room R3 is hindered. As a result, air conditioning efficiency decreases when the work room R3 is maintained at a predetermined target temperature, and the work room R3 is less likely to be maintained at the target temperature. The same applies to air conditioning of the other front rooms R2 and R4 and work room R5.

In contrast, in the first embodiment, the duct shafts DS1 and DS2 of the clean unit U1 include those that do not communicate with the chamber C1. In the example in FIG. 2, neither of the duct shafts DS1 and DS2 communicates with the chamber C1. The same applies to the duct shafts DS3 and DS4 of the other clean unit U2. Accordingly, occurrence of a closed loop flow can be prevented. Therefore, air conditioning efficiency of the work rooms R3 and R5 and the front rooms R2 and R4 is improved.

Next, processing during normal use of the clean room facility 100 and processing during sterilization will be described in order. The processing during normal use will be described mainly with reference to FIG. 2.

### Processing During Normal Use

During the normal use of the clean room facility 100, in addition to the air handling unit 30, the fan filter units 1 to 8 on the air supply side and the fan filter units 11 to 18 on the exhaust side are driven in a predetermined manner. The damper 71 on the air inlet side of the chamber C1 is in an open state, and the large room R1 and the chamber C1 communicate with each other via the damper 71 (the same applies to another damper 72). The damper 81 provided on the duct shaft DS1 is in an open state, and the duct shaft DS1 and the large room R1 communicate with each other via the damper 81 (the same applies to another dampers 82 to 84). Thus, when air conditioning of the clean room such as the work rooms R3 and R5 and the front rooms R2 and R4 is performed, the dampers 71 and 72 (first damper) and the dampers 81 to 84 (second damper) are maintained in the open state.

For example, a part of the air guided from the large room R1 to the chamber C1 via the damper 71 is guided to the work room R3, and a part of the air in the work room R3 is discharged to the large room R1 sequentially through the duct shaft DS1 and the damper 81. As described above, since the duct shaft DS1 does not communicate with the chamber C1, a closed loop flow of air hardly occurs. Therefore, air conditioning of the work room R3 can be performed with high efficiency. The same applies to the air conditioning of the other front rooms R2 and R4 and work room R5.

### Processing During Sterilization

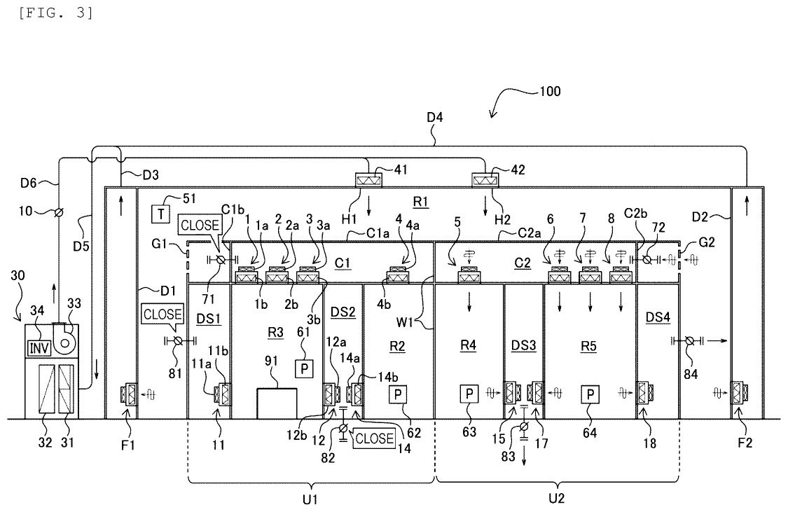

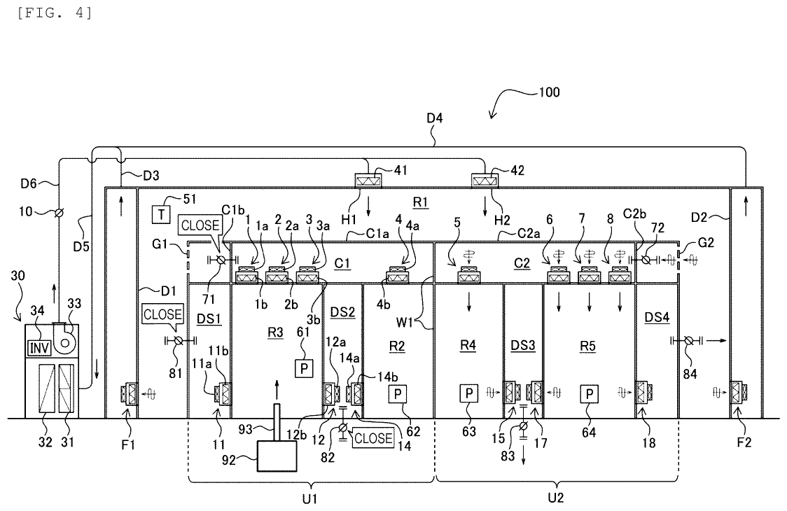

For example, there is a possibility that a sample is spilled when a person is working in the work room R3. In order to maintain a clean working environment, it is desirable to periodically sterilize the clean room. Therefore, processing illustrated in FIGS. 3 and 4 is appropriately performed during sterilization.

FIG. 3 is a diagram illustrating an example of processing during sterilization of the work room R3 and the front room R2.

A sterilization gas generator 91 illustrated in FIG. 3 is a device that generates a predetermined sterilization gas (hydrogen peroxide gas or the like). For example, the sterilization gas generator 91 is disposed in the work room R3, when the sterilization gas generator 91 is driven with each door closed, the sterilization gas is generated from the sterilization gas generator 91.

Since the chambers C1 and C2 are partitioned by the wall W1, for example, the sterilization gas generated by the sterilization gas generator 91 does not enter the adjacent chamber C2 from the work room R3 via the chamber C1. Therefore, even during the sterilization of the work room R3 or the front room R2, there is no particular problem even if the fan filter units 15 to 18 on the air supply side or the fan filter units 15 to 18 on the exhaust side of the other front room R4 and work room R5 are continuously driven, and there is no particular problem even if the dampers 72, 83, and 84 remains in an open state.

In contrast, during the sterilization of the work room R3 and the front room R2, the fan filter units 1 to 4 on the air supply side in the chamber C1 are maintained in a stopped state, and the fan filter units 11 to 14 that exhaust air from the work room R3 and the front room R2 are also maintained in the stopped state. When the work room R3 and the front room R2 (a plurality of clean rooms) are sterilized using the sterilization gas, the damper 71 (the first damper) and the dampers 81 and 82 (the second damper) are maintained in a closed state.

For example, the sterilization gas filling the work room R3 flows into the chamber C1 via the fan filter units 1 to 3 on the air supply side in the stopped state, and further flows into the front room R2 via the remaining fan filter unit 4 on the air supply side. In addition, the sterilization gas filling the work room R3 flows into the duct shaft DS1 via the fan filter unit 11 on the exhaust side in the stopped state, and also flows into the duct shaft DS2 via the fan filter unit 12 and the like. In addition, the sterilization gas flows into the front room R2 from the duct shaft DS2 via the fan filter unit 14 on the exhaust side. Thus, by driving the sterilization gas generator 91 in one clean room (the work room R3 in the example in FIG. 3), it is possible to collectively sterilize the work room R3, the front room R2, and the duct shafts DS1 and DS2 having the common chamber C1.

As described above, since the damper 71 on the air inlet side of the chamber C1 is in the closed state, the large room R1 and the chamber C1 are blocked by the damper 71. Therefore, it is possible to prevent the sterilization gas from leaking from the chamber C1 to the large room R1. Since the damper 81 provided on the duct shaft DS1 is also in the closed state, the large room R1 and the duct shaft DS1 are blocked by the damper 81. Therefore, it is possible to prevent the sterilization gas from leaking into the large room R1 through the duct shaft DS1. Similarly, it is also possible to prevent the sterilization gas from leaking to the large room R1 through another duct shaft DS2.

When the first clean unit U1 is sterilized, the sterilization gas generator 91 may be disposed in the front room R2 instead of the example in FIG. 3. The sterilization gas generator 91 may be disposed in each of the work room R3 and the front room R2. In short, when a plurality of clean rooms are sterilized, the sterilization gas generator 91 may be disposed in at least one of the plurality of clean rooms.

Even during the sterilization of the work room R3 and the front room R2, the air handling unit 30 and the fans F1 and F2 can be continuously driven. Accordingly, the large room R1 can be maintained at a predetermined temperature even during sterilization. In addition, for example, when the sterilization gas generator 91 is disposed in the work room R3, regarding air conditioning of the front room R4 or the work room R5 having the chamber C2 different from the chamber C1 above the ceiling of the work room R3, the fan filter units 5 to 8, and 15 to 18 can be continuously driven even during the sterilization of the work room R3 or the like. Therefore, work such as sample adjustment can be continuously performed in the front room R4 and the work room R5.

FIG. 4 is a diagram illustrating another example of processing during sterilization of the work room R3 and the front room R2.

As illustrated in FIG. 4, a sterilization gas generator 92 may be disposed outside the work room R3 (inside the large room R1 or outside the large room R1), and sterilization gas may be supplied from the sterilization gas generator 92 to the work room R3 via a hose 93. That is, when a plurality of clean rooms such as the work room R3 and the front room R2 are sterilized, the sterilization gas may be supplied from the sterilization gas generator 92 to at least one of the plurality of clean rooms from the outside via the hose 93. Even with such a configuration, a similar effect as in a case of FIG. 3 is achieved.

In addition to the hose 93 illustrated in FIG. 4, an exhaust hose (not illustrated) for exhausting air from the work room R3 may be connected to the sterilization gas generator 92. In this case, for example, air rendered harmless by catalyst gas (air in which a concentration of the sterilization gas is equal to or lower than a predetermined value) may be exhausted via the exhaust hose (not illustrated). The sterilization gas or the like may be circulated through a predetermined pipe or duct instead of the hose.

In addition, a decomposition device (not illustrated) including a predetermined catalytic filter (not illustrated) that renders the sterilization gas harmless may be separately provided. A catalytic filter may be provided downstream of the dampers 81 and 82 on the exhaust side, and a concentration of the sterilization gas may be reduced by the catalyst.

### Effects

According to the first embodiment, for example, since the duct shaft DS1 (see FIG. 2) does not communicate with the chamber C1, it is possible to prevent air from circulating (causing a closed loop) through the duct shaft DS1 and the chamber C1. Therefore, air conditioning of the work room R3 can be appropriately performed, and air conditioning efficiency when cooling the work room R3 is improved. The same applies to the air conditioning of the other front rooms R2 and R4 and work room R5.

For example, by providing the duct shaft DS1 between the work room R3 and the large room R1, the damper 81 can be provided on the duct shaft DS1. Therefore, by closing the damper 81 at the time of sterilization of the work room R3 or the like, it is possible to prevent the sterilization gas from leaking into the large room R1. By installing the fan filter unit 11 on the exhaust side inside the duct shaft DS1, the fan filter unit 11 can be made invisible from the large room R1. Accordingly, design of the clean room facility 100 is improved.

Since the front rooms R2 and R4 and the work rooms R3 and R5 are provided inside the large room R1, for example, it is easy to add a new clean room or change a layout inside the large room R1.

### First Modification of First Embodiment

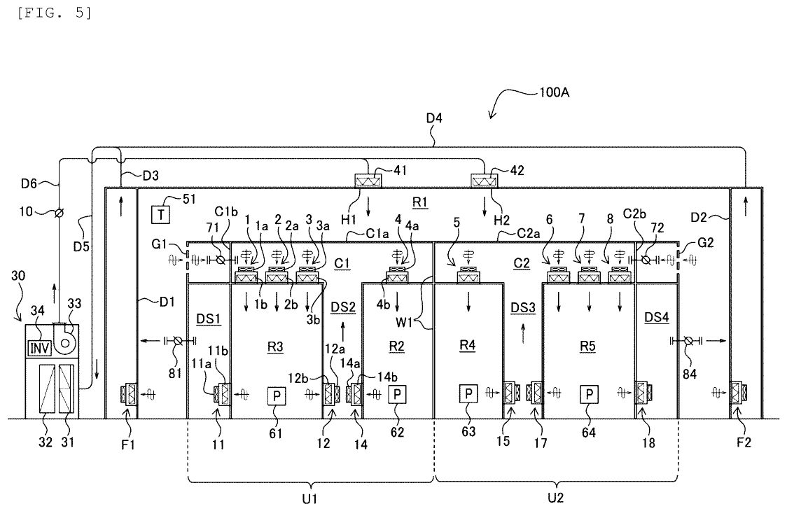

FIG. 5 is a schematic cross-sectional view illustrating a clean room facility 100A according to a first modification of the first embodiment.

The clean room facility 100A illustrated in FIG. 5 is different from the first embodiment (see FIG. 2) in that the duct shaft DS2 communicates with the chamber C1 and the duct shaft DS3 communicates with the other chamber C2. The clean room facility 100A illustrated in FIG. 5 is different from the first embodiment in that no damper is provided on the duct shafts DS2 and DS3, and neither of the duct shafts DS2 and DS3 communicates with the large room R1. Other configurations are similar as those of the first embodiment, and thus description of overlapping portions will be omitted.

As illustrated in FIG. 5, for example, in the first clean unit U1, the duct shaft DS2 communicates with the chamber C1, but another duct shaft DS1 does not communicate with the chamber C1. Therefore, it is possible to prevent occurrence of a closed loop flow of air at least in a flow path through the duct shaft DS1. Similarly, in a flow path through the duct shaft DS4 of the second clean unit U2, a closed loop flow of air hardly occurs. Therefore, although air conditioning efficiency is slightly lower than that of the first embodiment, the air conditioning of the work room R3 and the like can be appropriately performed.

The air flowing into the duct shaft DS2 from the work room R3 is returned to the chamber C1 through the duct shaft DS2. Therefore, the air with high cleanliness supplied to the chamber C1 can be reused.

A combination of communication and non-communication between a predetermined duct shaft and the chambers C1 and C2 is not limited to the example in FIG. 5. For example, the duct shaft DS1 may communicate with the chamber C1, another duct shaft DS4 may communicate with the chamber C2, and the remaining duct shafts DS2 and DS3 may not communicate with the chambers C1 and C2. Even with such a configuration, air conditioning of the work room R3 and the like can be appropriately performed. In short, at least one duct shaft that does not communicate with the chambers C1 and C2 among the plurality of duct shafts DS1, DS2, DS3, and DS4 may be included.

In the example in FIG. 5, an example is illustrated in which the damper is not installed on the duct shafts DS2 and DS3 communicating with the chamber C1, but the invention is not limited thereto. That is, a damper (not illustrated) may be installed on each of the duct shafts DS2 and DS3. In this case, for example, a part of the air flowing into the duct shaft DS2 from the work room R3 is returned to the chamber C1 through the duct shaft DS2, and the remaining air is guided to the large room R1 via a damper (not illustrated) in an open state. Even with such a configuration, clean air can be reused while appropriately performing air conditioning of each clean room such as the work room R3.

### Second Modification of First Embodiment

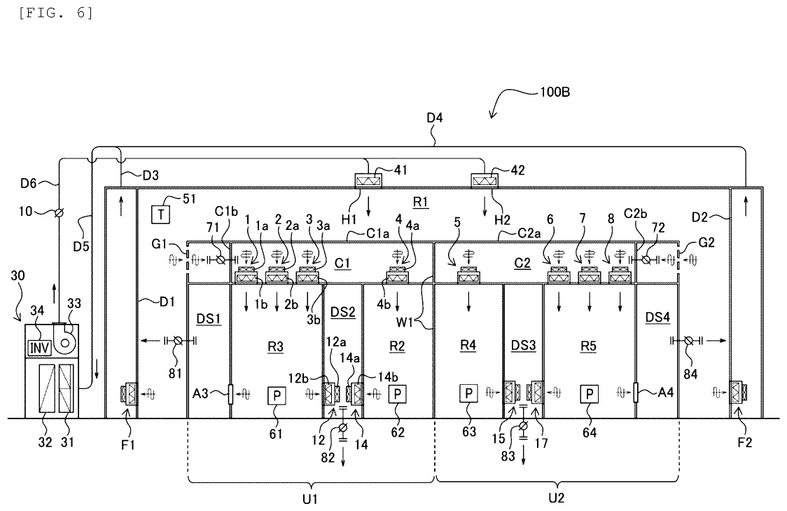

FIG. 6 is a schematic cross-sectional view illustrating a clean room facility 100B according to a second modification of the first embodiment.

The clean room facility 100B illustrated in FIG. 6 is different from the first embodiment in that the duct shaft DS1 is not provided with a fan filter unit on the exhaust side and an opening A3 is provided instead (the same applies to another duct shaft DS4) Other configurations are similar as those of the first embodiment, and thus description of overlapping portions will be omitted.

As illustrated in FIG. 6, the opening A3 is provided on the work room R3 side of the duct shaft DS1. Then, air is exhausted from the work room R3 to the large room R1 sequentially through the opening A3, the duct shaft DS1, and the damper 81 (open state). The same applies to exhaust through the duct shaft DS4. Even with such a configuration, a similar effect as that of the first embodiment is achieved. Positions of openings A3 and A4 are not limited to the example in FIG. 6, and some or all of the exhaust fans 11a to 18a (see FIG. 2) described in the first embodiment may be omitted, and openings may be provided at omitted positions. In addition, a filter may be appropriately installed in each of these openings.

### Second Embodiment

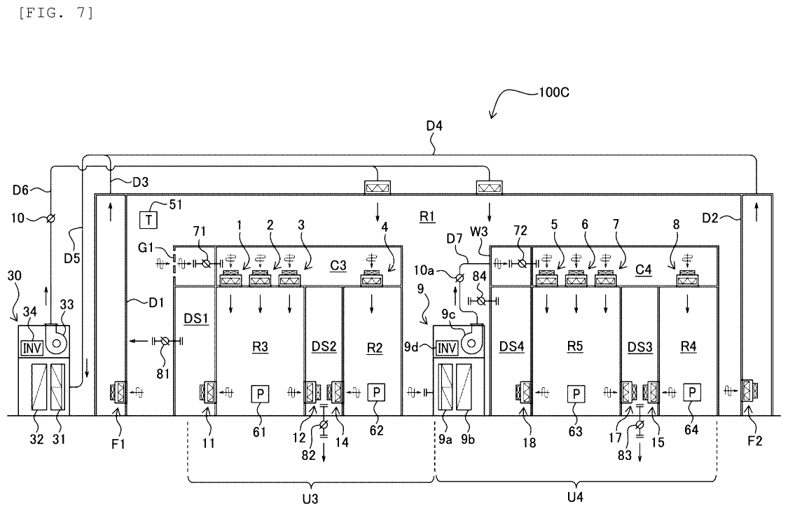

A second embodiment is different from the first embodiment in that a first clean unit U3 (see FIG. 7) having a predetermined chamber C3 and a second clean unit U4 (see FIG. 7) having another chamber C4 are provided in a state of being separated from each other. The second embodiment is different from the first embodiment in that an air handling unit 9 (see FIG. 7) that individually performs air conditioning on the second clean unit U4 is provided. Other configurations are similar as those of the first embodiment. Therefore, parts different from those of the first embodiment will be described, and description of repeated parts will be omitted.

FIG. 7 is a schematic cross-sectional view illustrating a clean room facility 100C according to the second embodiment.

As illustrated in FIG. 7, the clean room facility 100C includes the first clean unit U3, the second clean unit U4, and the air handling unit 9 (second air handling unit). The first clean unit U3 includes the work room R3 and the front room R2 as a plurality of clean rooms having the common chamber C3. Similarly, the second clean unit U4 includes the work room R5 and the front room R4 as a plurality of clean rooms having the common chamber C4. The first clean unit U3 and the second clean unit U4 are provided inside the large room R1 (predetermined room).

In the example in FIG. 7, the first clean unit U3 and the second clean unit U4 are separated from each other, but for example, as illustrated in FIGS. 2, 5, and 6, even in a configuration in which the clean units are partitioned by the wall W1, the air handling unit 9 (air conditioner) can be installed.

Since the configuration of the first clean unit U3 is similar as that of the first clean unit U1 (see FIG. 2) described in the first embodiment, the description thereof will be omitted. The second clean unit U4 has substantially the same configuration as the first clean unit U3. The second clean unit U4 is different from the first clean unit U3 in that a grill is not provided upstream of the damper 72 and a wall W3 is provided instead. A downstream end of a duct D7 through which air cooled by the air handling unit 9 flows is inserted into the wall W3. The downstream end of the duct D7 may be directly connected to the damper 72.

The second clean unit U4 has an air conditioning load larger than that of the first clean unit U3. For example, when a heat generation amount of a device (not illustrated) installed in the work room R5 or the like is large, or when a set temperature (target temperature) of the work room R5 or the like is lower than that of the work room R3 or the like of the first clean unit U3, an air conditioning load of the second clean unit U4 is relatively large in many cases. Therefore, in the second embodiment, the air in the large room R1 is additionally cooled by the air handling unit 9 and then supplied to the second clean unit U4.

The air handling unit 9 is an air conditioner that suctions air in the large room R1 (predetermined room), adjusts a temperature, and supplies the temperature-adjusted air to the chamber C4 of the second clean unit U4. The air handling unit 9 includes a filter 9a, a cooling coil 9b, a fan 9c, and an inverter 9d. Since the configuration of the air handling unit 9 illustrated in FIG. 7 is similar as that of another air handling unit 30 for cooling the air in the large room R1, the description thereof will be omitted. Air suctioned into the air handling unit 9 from the large room R1 is cooled to a predetermined temperature, and the cooled air is guided to the chamber C4 sequentially through the duct D7 and the damper 72 (open state).

As illustrated in FIG. 7, a damper 10a is installed in the duct D7. For example, the damper 10a is set to a predetermined opening degree during a test operation of the air handling unit 9, and is maintained at the predetermined opening degree during a subsequent air conditioning operation. The opening degree of the damper 10a may be appropriately adjusted based on an air conditioning load of the front room R4 or the work room R5.

Regarding the first clean unit U3, the work room R3 and the front room R2 are maintained at a predetermined set temperature by supplying air in the large room R1 (that is, the air cooled by the air handling unit 30) as it is. Therefore, it is not particularly necessary to provide an air handling unit that performs individual air conditioning in the first clean unit U3.

In a configuration illustrated in FIG. 7, the air handling unit 9 is configured to suction the air in the large room R1, but the invention is not limited thereto. For example, the air handling unit 9 may be configured to selectively suction air in the large room R1 and air in the work room R5 based on temperature information of the large room R1 and temperature information of the work room R5. As a specific example, in a case where the cooling operation is performed by the air handling unit 9, when a temperature of the work room R5 is lower than a temperature of the large room R1, the air handling unit 9 suctions the air in the work room R5. When the temperature of the large room R1 is lower than the temperature of the work room R5, the air handling unit 9 suctions the air in the large room R1. Accordingly, an air conditioning load of the air handling unit 9 is reduced, and thus energy saving can be achieved. In the above-described configuration, when the temperature of the large room R1 and the temperature of the work room R5 are substantially equal to each other, a target when the air handling unit 9 suctions air may be either the large room R1 or the work room R5.

In the above-described configuration, if the damper is not provided in the duct connecting the air handling unit 9 and the work room R5, sterilization gas flows into the air handling unit 9 via the work room R5 when the work room R5 is sterilized. In order to avoid such a situation, a damper may be provided in the duct connecting the air handling unit 9 and the work room R5. By providing, for example, a non-leak damper as such a damper, it is possible to effectively prevent the sterilization gas from flowing into the air handling unit 9.

### Effects

According to the second embodiment, the air further cooled by the air handling unit 9 is supplied to the chamber C4 of the second clean unit U4. Therefore, even when an air conditioning load of the second clean unit U4 is larger than that of the first clean unit U3, air conditioning suitable for a use environment of the second clean unit U4 can be individually performed. Air conditioning of the large room R1 and the first clean unit U3 can be appropriately performed by the other air handling unit 30.

### Modification

Although the clean room facility 100 and the like according to the invention are described in the embodiments, the invention is not limited thereto, and various modifications can be made.

For example, in each embodiment, the configuration is described in which a plate member (not illustrated) is not particularly provided in a gap between the ceiling of the large room R1 (see FIG. 2) and the upper plate C1a of the chamber C1, but the invention is not limited thereto. That is, in order to prevent dust from accumulating on the upper plate C1a of the chamber C1 and also taking into consideration of an appearance of an upper side of the chamber C1, a plate member (not illustrated) that connects the ceiling of the large room R1 and the upper plate C1a of the chamber C1 in the vertical direction may be provided along an edge of the chamber C1. Such a configuration is also in a matter that the first clean unit U1 and the like are present inside the large room R1.

In the embodiments, a case is described in which a predetermined gap is provided between the ceiling of the large room R1 and the upper plate C1a of the chamber C1 and between the ceiling of the large room R1 and the upper plate C2a of the chamber C2, but the invention is not limited thereto. For example, the upper plates C1a and C2a may be integrated with the ceiling of the large room R1. A side wall of the first clean unit U1 or the like may be integrated with a part of a side wall of the large room R1. These configurations are also in a matter that the first clean unit U1 and the like are present inside the large room R1.

In the embodiments, a case is described in which the exhaust destination from the clean room such as the work room R3 is the large room R1, but the invention is not limited thereto. For example, the exhaust destination from the clean room such as the work room R3 may be a space outside the clean room facility 100.

In the embodiments, a case is described in which the number of clean room units (the first clean unit U1 and the second clean unit U2) is two, but the invention is not limited thereto. That is, at least one clean room unit may be provided inside the large room R1 (predetermined room).

In the embodiments, a case is described in which the dampers 81 to 84 (second damper) are provided so as to correspond to the duct shafts DS1, DS2, DS3, and DS4, but the invention is not limited thereto. For example, at least a part of the dampers 81 to 84 may be omitted, and a predetermined duct shaft and the large room R1 may communicate with each other via an exhaust port (second exhaust port: not illustrated). In such a configuration, at the time of sterilization of the clean room, an operator may cover the exhaust port so that the sterilization gas does not flow into the large room R1 via the exhaust port.

In the first embodiment, a case is described in which the sterilization gas generators 91 and 92 (see FIGS. 3 and 4) that generate the sterilization gas are provided, but the invention is not limited thereto. For example, a device having a function of aeration by a catalyst or the like in addition to a function of dehumidification or predetermined gas generation may be provided. The sterilization gas generators 91 and 92 may also have this function.

In the embodiments, a case is described in which air in the large room R1 (see FIG. 2) is guided to the air handling unit 30, and air cooled by the air handling unit 30 is returned to the large room R1, but the invention is not limited thereto. For example, an outdoor air handling unit (not illustrated) of a clean room facility may take in outside air, and guide the air cooled by the air handling unit to the large room R1. In this case, the exhaust destination from the predetermined clean room may be outside.

Although a layout of the clean room facility 100 or the like described in the embodiments is an example, the following configuration may be adopted in other layouts. That is, the clean room facility may include a duct shaft provided in a gap between the clean rooms among the plurality of clean rooms and/or provided in a gap between a predetermined clean room and the large room R1 (predetermined room). One or a plurality of duct shafts may be provided outside a side wall of at least one clean room. In such a configuration, the one or more duct shafts include the duct shaft that does not communicate with the chamber. Accordingly, air can be exhausted from a predetermined clean room to the large room R1 or the like through the duct shaft that does not communicate with the chamber, so that air can be prevented from circulating in a closed loop.

As illustrated in FIG. 1, the duct shafts DS1, DS2, DS3, and DS4 may be arranged on a straight line along a predetermined side wall facing the large room R1. Accordingly, when the operator opens a maintenance cover (not illustrated) of each of the duct shafts DS1, DS2, DS3, and DS4 to expose the fan filter units 11 to 18, the operator can sequentially perform an operation along the side wall, and thus the operation at the time of maintenance is easily performed.

In a configuration of FIG. 5 (first modification of the first embodiment), for example, a perforated plate or a grating may be provided at the upper end (downstream end of an air flow) of the duct shaft DS2. Such a configuration is also in the matter that the duct shaft DS2 communicates with the chamber C1.

In the second embodiment (see FIG. 7), a case is described in which the air handling unit 9 individually performs air conditioning of the second clean unit U4, but the invention is not limited thereto. That is, two air handling units 9 (second air handling units) may be installed inside the large room R1 so as to correspond one-to-one to the first clean unit U3 and the second clean unit U4. Then, the air in the large room R1 may be cooled by each air handling unit 9, and the cooled air may be guided to the chambers C3 and C4. According to such a configuration, even when set temperatures of the first clean unit U3 and the second clean unit U4 are different from that of the large room R1, the air conditioning can be assisted by the air handling units 9.

For example, a duct shaft may be provided in a gap between the side wall of the large room R1 and a predetermined clean room. Such a configuration is also in a matter that the duct shaft is provided in the gap between the clean room and the large room R1. The duct shaft may not communicate with the chamber C1, and air may be exhausted from the clean room to an outside of the large room R1 through the duct shaft. Even with such a configuration, since circulation of air in a closed loop through the duct shaft is prevented, air conditioning efficiency is improved.

In the embodiments, the configuration is described in which the damper 71 (see FIG. 2) is hidden by the grill G1 and another damper 72 (see FIG. 2) is hidden by the grill G2 when viewed from an inside of the large room R1, but the invention is not limited thereto. That is, the grills G1 and G2 may be omitted as appropriate, and the dampers 71 and 72 may be exposed to the large room R1.

In the embodiments, the configuration is described in which the damper 81 is provided on the large room R1 side with respect to the duct shaft DS1 (a main body portion of the damper 81 is exposed to the large room R1), but the invention is not limited thereto. For example, the damper 81 may be installed inside the duct shaft DS1. Accordingly, the damper 81 is hidden when viewed from the inside of the large room R1, so that design is improved. The same applies to the other dampers 82 to 84.

When an air conditioning load of a plurality of clean rooms such as the front rooms R2 and R4 and the work rooms R3 and R5 is relatively large, the following configuration may be adopted. That is, it is preferable that the air outlets H1 and H2 (first air outlets: see FIG. 1) for air whose temperature is adjusted by the air handling unit 30 (first air handling unit) are provided upstream in an air flow direction with respect to the air inlets (first air inlets: gaps of the grills G1 and G2) near the chambers C1 and C2. Accordingly, conditioned air blown out from the air outlets H1 and H2 is guided to the chamber C1 via the air inlet as it is, so that the cooled air can be sent to the front rooms R2 and R4 and the work rooms R3 and R5.

For example, the damper 81 (second damper) or the second exhaust port (not illustrated) that guides air exhausted from the duct shaft DS1 (see FIG. 2) to the large room R1 (predetermined room) may be provided in the duct shaft DS1. Further, the duct D1 (see FIG. 1) may be provided with the air inlet H3 (second air inlet: see FIG. 1) through which air is suctioned from the large room R1 toward the air handling unit 30 (first air handling unit). In such a configuration, the air inlet H3 (second air inlet) is preferably provided downstream in an air flow direction with respect to the damper 81 (second damper) or the second exhaust port. Accordingly, the air that absorbs heat in the clean room is guided to the duct D1 as it is sequentially through the damper 81 and the like and the air inlet H3, so that a rise in air temperature in the large room R1 can be prevented.

In the embodiments, the configuration is described in which the chambers C1 and C2 (see FIG. 2) are partitioned by the wall W1, but the invention is not limited thereto. That is, the chambers C1 and C2 may be a single common space without providing the wall W1.

In the embodiments, a case is described in which the clean rooms such as the work rooms R3 and R5 and the front rooms R2 and R4 are used as positive pressure rooms, but the clean rooms may m be used as negative pressure rooms depending on the application.

Although a case in which the clean room facility 100 and the like are used for cell culture processing or pharmaceutical manufacturing is described in the embodiments, the invention is not limited thereto. For example, the embodiments can be applied to various fields such as manufacturing of semiconductors, precision machines, and liquid crystal panels, food industry, cosmetic industry, and experiments using radioactive substances.

The embodiments have been described in detail in order to describe the invention in an easy-to-understand manner, and are not limited to necessarily including all the described configurations. In addition, it is possible to add, delete, or replace some of the configurations of the embodiments with other configurations.

Mechanisms and configurations described above indicate what is considered to be necessary for explanation, and not all mechanisms and configurations are necessarily shown on a product.

### REFERENCE SIGNS LIST

1, 2, 3, 4, 5, 6, 7, 8: fan filter unit
1a, 2a, 3a, 4a: air supply fan
9: air handling unit (second air handling unit)
11, 12, 13, 14, 15, 16, 17, 18: fan filter unit
11a, 12a, 13a, 14a: exhaust fan
30: air handling unit (first air handling unit)
71, 72: damper (first damper)
81, 82, 83, 84: damper (second damper)
91, 92: sterilization gas generator
93: hose
100, 100A, 100B, 100C: clean room facility
C1, C2: chamber
D1, D2: duct
DS1, DS2, DS3, DS4: duct shaft
G1, G2: grill (first air inlet)
H1, H2: air outlet (first air outlet)
H3, H4: air inlet (second air inlet)
R1: large room (predetermined room)
R2, R4: front room (clean room)
R3, R5: work room (clean room)
U1, U3: first clean unit (clean unit)
U2, U4: second clean unit (clean unit)

1. A clean room facility comprising:
a plurality of clean rooms provided inside a predetermined room;
a first air handling unit configured to adjust a temperature of air supplied to the predetermined room;
a chamber provided as one common space above a ceiling of the plurality of clean rooms and configured to guide air from the predetermined room;
an air supply fan provided in each of the plurality of clean rooms and configured to supply air from the chamber to the clean room; and
a duct shaft provided outside a side wall of at least one of the clean rooms, wherein
among the duct shafts, the duct shafts facing the predetermined room include a plurality of duct shafts whose upper ends are closed so as not to communicate with the chamber, and further, provided with maintenance covers,
air is guided from the clean room to the duct shaft not communicating with the chamber, and is exhausted through the duct shaft, and
an exhaust destination when the air is exhausted from the clean room through the duct shafts is the predetermined room.

2. (canceled)

3. The clean room facility according to claim 1, wherein
a ceiling of the predetermined room is provided with a first air outlet through which air whose temperature is adjusted by the first air handling unit is blown out,
a first air inlet through which the air flowing from the predetermined room toward the chamber is suctioned is provided near the chamber, and
the first air outlet is provided upstream of the first air inlet in an air flow direction.

4. The clean room facility according to claim 1, wherein
the duct shaft is provided with a second damper or a second exhaust port that guides air exhausted from the duct shaft to the predetermined room,
a duct is provided with a second air inlet through which air from the predetermined room toward the first air handling unit is suctioned, and
the second air inlet is provided downstream of the second damper or the second exhaust port in an air flow direction.

5. The clean room facility according to claim 1, wherein
the duct shafts include a duct shaft communicating with the chamber.

6. The clean room facility according to claim 1, further comprising:
a first damper configured to switch between communication and interruption between the predetermined room and the chamber; and
a second damper configured to switch between communication and interruption between the duct shaft and the predetermined room, wherein
the first damper and the second damper are maintained in an open state when air conditioning of the plurality of clean rooms is performed.

7. The clean room facility according to claim 1, further comprising:
a first damper configured to switch between communication and interruption between the predetermined room and the chamber; and
a second damper configured to switch between communication and interruption between the duct shaft and the predetermined room, wherein
the first damper and the second damper are maintained in a closed state when sterilization of the plurality of clean rooms is performed using sterilization gas.

8. The clean room facility according to claim 7, further comprising:
a sterilization gas generator disposed in at least one of the plurality of clean rooms when sterilizing the plurality of clean rooms.

9. The clean room facility according to claim 7, further comprising:
a sterilization gas generator configured to supply sterilization gas to at least one of the plurality of clean rooms from outside via a hose when sterilizing the plurality of clean rooms.

10. The clean room facility according to claim 1, wherein
at least one clean unit including the plurality of clean rooms having a common chamber is provided inside the predetermined room, and
a second air handling unit configured to suction air in the predetermined room to adjust a temperature of the air, and supply the temperature-adjusted air to the chamber.

11. The clean room facility according to claim 1, wherein
the predetermined room is a normal room whose cleanliness is not managed.
